(19) United States
(12) Patent Application Publication
(10) Pub. No.: US 2024/0237983 A1
(43) Pub. Date: Jul. 18, 2024

(54) DEVICES AND METHODS FOR TREATING HEART FAILURE

(71) Applicant: Corvia Medical, Inc., Tewksbury, MA (US)

(72) Inventors: Stephen J. FORCUCCI, Winchester, MA (US); Matthew J. FINCH, Reading, MA (US); Alexander T. MADDEN, Somerville, MA (US); Michael W. SUTHERLAND, Groton, MA (US); Edward I. MCNAMARA, Chelmsford, MA (US)

(21) Appl. No.: 18/416,440

(22) Filed: Jan. 18, 2024

Related U.S. Application Data
(60) Provisional application No. 63/480,455, filed on Jan. 18, 2023.

(51) Int. Cl. A61B 17/11; A61B 17/00

(52) U.S. Cl. CPC A61B 17/11; A61B 2017/00243; A61B 2017/00292; A61B 2017/1139

(57) ABSTRACT

A device for implanting into an atrial septum of a patient. In embodiments, the device has a core region to be disposed in an atrial septum opening; a distal retention portion adapted to engage tissue on a left atrial side of the septal wall; a proximal retention portion adapted to engage tissue on a right atrial side of the septal wall; and a retrieval portion comprising a plurality of retrieval members each with a connector at its proximal end. The device has a delivery configuration and a deployed configuration. The core region, distal retention portion, and proximal retention portion each has a smaller diameter in the delivery configuration than in the deployed configuration. In the deployment configuration, the proximal end of the retrieval member is disposed radially outward from the central opening of the core region.

### CROSS-REFERENCE TO RELATED APPLICATION

This application claims priority to and the benefit of U.S. Provisional Application No. 63/480,455, filed Jan. 18, 2023, the entirety of which is incorporated herein by reference.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The present teachings relate to devices and methods of use thereof for treating heart failure. An aspect of the present teachings relates to a device that can be used to change (e.g., reduce) the blood pressure in a heart chamber, for example, by creating a shunt, and optionally regulating the flow of blood through the shunt in order to enhance the therapeutic effect of the shunt. The present teachings further relate to a method of utilizing such a device, for example, in treating congestive heart failure and its related conditions, for example, acute cardiogenic pulmonary edema caused by an elevated pressure in a left side chamber in the heart.

### BACKGROUND

Congestive heart failure (CHF) is a condition that affects millions of people worldwide. CHF results from a weakening or stiffening of the heart muscle that commonly is caused by myocardial ischemia (due to, e.g., myocardial infarction) or cardiomyopathy (e.g., myocarditis, amyloidosis). CHF causes a reduced cardiac output and inadequate blood to meet the needs of body tissues. Heart failure with preserved ejection fraction (HFpEF) is a condition among patients with heart failure that occurs when the muscles of the left atrium (LA) and ventricle (LV) become stiffer and are unable to relax normally. As a result, blood cannot easily exit the LA into the LV with each heartbeat, causing high pressure inside the lungs and left heart chambers.

Treatments for CHF include: (1) pharmacological treatments, (2) assisting systems, and (3) surgical treatments. Pharmacological treatments, e.g., with diuretics, are used to reduce the workload of a heart by reducing blood volume and preload. While pharmacological treatments can improve quality of life, they have little effect on survival. Assisting devices, e.g., mechanical pumps, are used to reduce the load on a heart by performing all or part of the pumping function normally done by the heart. However, in a chronic ischemic heart, high-rate pacing may lead to an increased diastolic pressure, calcium overload, and damages to the muscle fibers. There are at least three surgical procedures for treating a heart failure: (1) heart transplant, (2) dynamic cardiomyoplasty, and (3) the Batista partial left ventriculectomy. These surgical treatments are invasive and have many limitations.

CHF is generally classified into systolic heart failure (SHF) or diastolic heart failure (DHF). In SHF, the pumping action of a heart is reduced or weakened. A normal ejection fraction (EF), the volume of blood ejected out of the left ventricle (stroke volume) divided by the maximum volume remaining in the left ventricle at the end of the diastole or relaxation phase, is greater than 50%. In a systolic heart failure, EF is decreased to less than 50%. A patient with SHF may have an enlarged left ventricle because of cardiac remodeling developed to maintain an adequate stroke-volume. This pathophysiological phenomenon is often associated with an increased atrial pressure and an increased left ventricular filling pressure.

DHF is a heart failure without any major valve disease even though the systolic function of the left ventricle is preserved. Generally, DHF is a failure of the ventricle to adequately relax and expand, resulting in a decrease in the stroke volume of the heart. Presently, there are very few treatment options for patients suffering from DHF. DHF afflicts between 30% and 70% of patients with CHF.

Devices to treat elevated left atrial pressure have been described. For example, U.S. Pat. Nos. 8,740,962 and 8,460,372 both describe prostheses that may be implanted in an opening in the septal wall of the heart to provide a shunt or channel permitting blood to flow from the left atrium into the right atrium. These devices collapse to a smaller configuration for delivery to the heart via a catheter and expand to a larger configuration (e.g., through self-expansion) upon deployment across an opening in the septal wall. Some of these devices have central cores with sufficient radial strength to maintain the patency of the septal wall opening and flexible anchors on both sides of the central core to contact the septal wall for atraumatic anchoring of the device. Some of these devices have retrieval legs and other features providing attachment points for delivery and/or retrieval for possible removal or redeployment.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1D show various views of an embodiment of a pressure regulating device.

FIG. 2 shows a flat layout view of an embodiment of a pressure regulating device.

FIG. 3 shows a flat layout view of a portion of an embodiment of a pressure regulating device.

FIG. 4 shows a perspective view of an embodiment of a tube after laser cutting to form a pressure regulating device.

FIG. 5 shows an embodiment of a pressure regulating device in a delivery configuration.

FIG. 6 shows an embodiment of a pressure regulating device as it is being deployed.

FIG. 7 shows an embodiment of a pressure regulating device as it is being released from a delivery system.

FIG. 8 shows a flat layout view of a portion of a pressure regulating device.

FIG. 9 shows an embodiment of a pressure regulating device as it is being withdrawn into a delivery system.

FIG. 10 shows a flat layout view of a portion of an embodiment of a pressure regulating device.

FIG. 11 shows an embodiment of a pressure regulating device as it is being withdrawn into a delivery system.

FIG. 12 shows a flat layout view of a portion of an embodiment of a pressure regulating device.

### DETAILED DESCRIPTION

The present teachings are described more fully herein with references to the accompanying drawings, which show certain embodiments of the present teachings. The present teachings may, however, be embodied in many different forms and should not be construed as being limited to the embodiments set forth herein. Rather, these embodiments are provided to illustrate various aspects of the present teachings. Like numbers refer to like elements throughout.

The present teachings provide a device and methods of use thereof. For example, the device can be used to regulate the pressure in a heart chamber. Specifically, the device can be used to (a) change an elevated chamber pressure and/or (b) prevent embolization from the right to left atria in a patient who suffers from CIF or has a Patent Foramen Ovale (PFO) or an Atrial Septal Defect (ASD) but needs a residual flow between the atria so as not to traumatize the heart hemodynamics.

As used herein, when terms “distal” and “proximal” are used to refer portion of the device, they refer to a device in its deployed configuration. The term “proximal” shall mean close to the operator (less into the body) and “distal” shall mean remote from the operator (further into the body). In positioning a medical device from a downstream access point, “distal” is more upstream and “proximal” is more downstream. As used in this application, unless otherwise indicated, the term “opening” refers to any anatomical anomalies such as PFO, ASD, VSD, or an anatomical feature (such as an opening in the septal wall) created for the purpose of creating a shunt. As used herein, “substantially” means plus or minus 10%.

As used herein, a “flat layout view” can refer to a view of the device in a flattened state and/or opened state such that no portions of the device are overlapping. For example, a flat layout view of a device comprising a generally tubular shape comprises a view of the device as though it is cut along its length and unfurled such that lays flat.

As explained in further detail below, various embodiments of the present teachings provide methods and devices for regulating the pressure in a heart chamber. In some embodiments, a medical device according to the present teachings includes an open core region and two retention portions. In some embodiments, the medical device is positioned through an opening in a septum, creating a shunt, for example, between the left and right atria. In some embodiments, the two retention portions of the medical device are disposed on the opposite sides of the septum. In some embodiments, a medical device according to the present teachings is extended into an elongated profile for a percutaneous delivery and resumes a preset profile in vivo after deployment.

An embodiment of the device in the present teachings has a distal retention portion configured to be positioned against the left atrial side of the septum, a proximal retention portion configured to be positioned against the right atrial side of the septum, and a core region disposed between the distal and proximal retention portions and configured to create a conduit for blood to flow through.

An embodiment of the device in the present teachings has an elongated configuration for delivering through a catheter delivery system and an expanded deployed configuration securing the device across the septum. In some embodiments, the device is configured to transition from a delivery configuration to a deployed configuration through self-expansion or mechanical actuations. In some embodiments, during deployment, both the distal and proximal retention portions of the device are delivered in radially contracted configurations and expand radially while the device contracts longitudinally. In some embodiments, the core region is delivered in a radially contracted configuration and expands radially during deployment. In certain embodiments, one or both of the distal and proximal retention portions of the device contract longitudinally during deployment.

In various embodiments, one of or both of the deployed distal and proximal retention portions has/have a generally flange-like profile. In various embodiments, the generally flange-like profile is made of a multiple segments or elements extending in a generally radial configuration from the core region. In some embodiments, the deployed distal retention portion is configured to be positioned against one side of the atrial septum. In some embodiments, the deployed proximal retention portion is configured to be positioned against one side of the atrial septum. In certain embodiments, each of the deployed distal retention portion and the deployed proximal retention portion are configured to be positioned against opposing sides of the atrial septum.

According to some embodiments, the deployed distal and proximal retention portions apply a compression force against the septum from both sides, thereby securing the device across the septum.

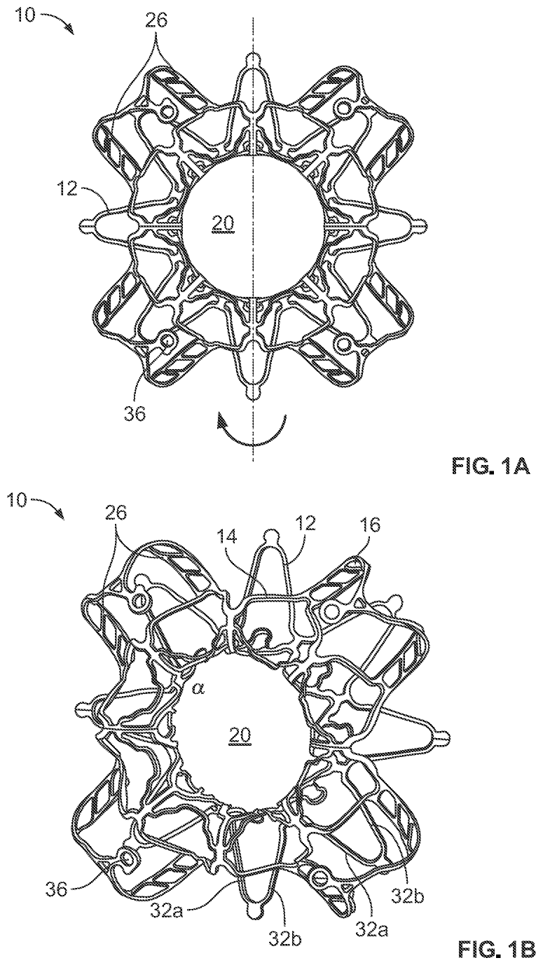

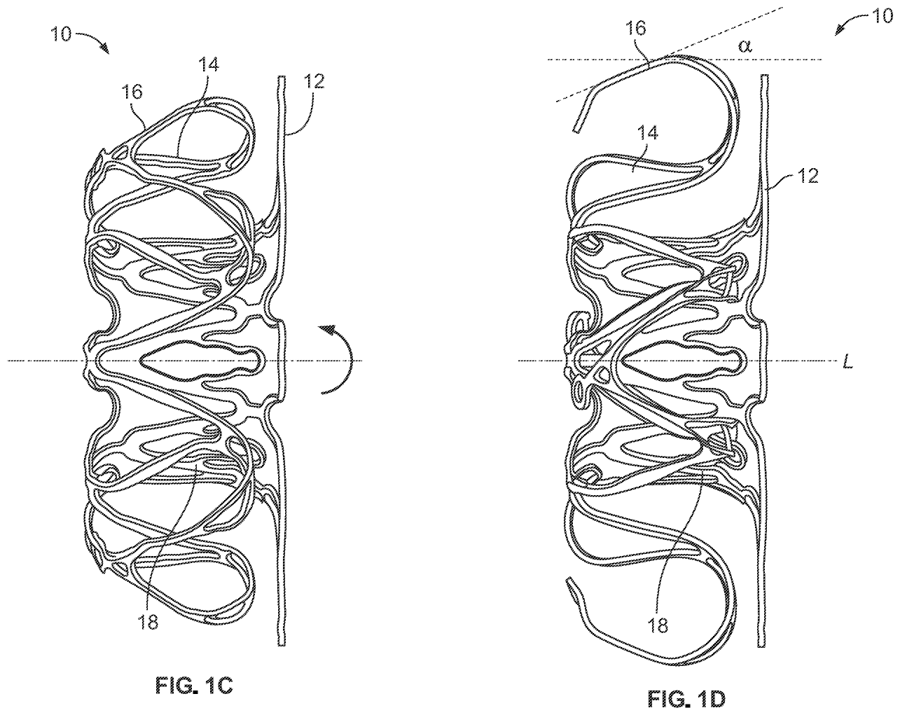

FIGS. 1A-1D show various views of the device (10) in a deployed configuration, in accordance with an embodiment described herein. FIGS. 1A and 1B show front views of an embodiment of a fully deployed pressure regulating device (10), looking from the proximal end side, with the view in FIG. 1B rotated 45° in the direction of the arrow shown in FIG. 1A. FIGS. 1C and 1D both show side views of the device (10), with the view in FIG. 1D rotated 45° in the direction of the arrow relative to FIG. 1C to show the proximal retention portion (14) and the retrieval portion (16) of the device (10). As shown in FIG. 1C, a distal retention portion (12) extends distally and radially outward from a proximal end of the core region (18), a proximal retention portion (14) extends proximally and radially outward from the core region (18), and a retrieval portion (16) extends proximally and radially inward from the distal end of the proximal retention portion (14).

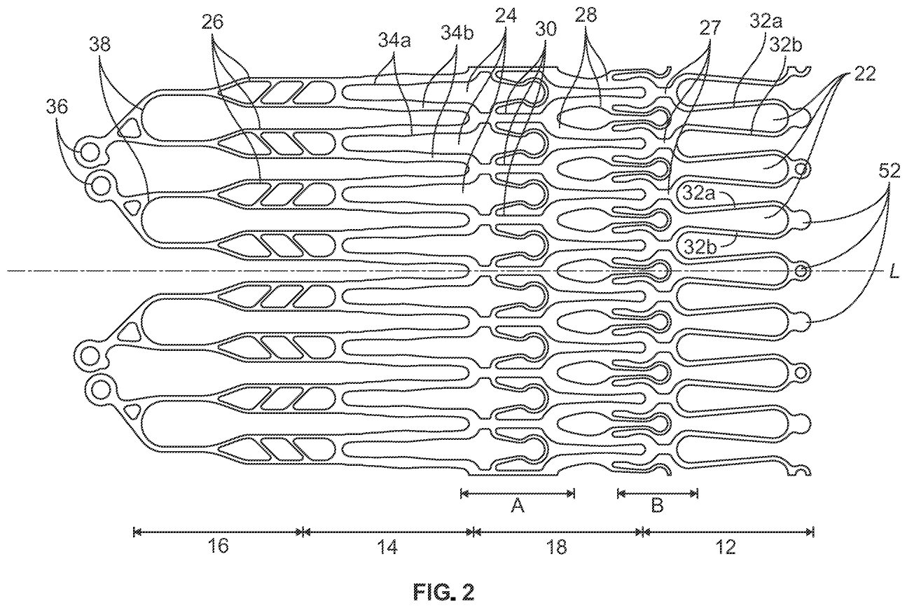

In the deployed configuration shown in FIGS. 1A-1D, the deployed core region (18) has a tubular profile with a central opening (20) that, when deployed through a septal wall of a heart, permits blood to flow through the device (10) from the left atrium to the right atrium, thus lowing the pressure in the left atrium. According to one embodiment of the present teachings, the core region (18) comprises a plurality of struts (28) joining adjacent struts at alternative ends and forming a continuous rounded zigzag pattern with hairpin turns at both the distal and proximal ends of the core region as illustrated in a flat layout view in FIG. 2. When deployed in vivo, the deployed core region (18) is configured to apply a radially outward force toward its surrounding septum, and as such, the deployed core region (18) enlarges the opening on the septal wall. In other embodiments, the fully deployed core region (18) has an outer diameter similar to the opening on the septal wall, so that it does not further extend radially outward upon deployment in vivo, and therefore does not apply a radially outward force toward the surrounding septum. In one embodiment, the strut width and thickness of the core region (18) could be configured to provide a desired chronic radial outward force when deployed.

Continuing referring to FIGS. 1A-1D, the core region (18) joins a distal retention portion (12) at its distal end. As shown in the figures, the distal retention portion (12) has a plurality of distal retention segments (22), each formed by two struts (32a, 32b). When the device (10) is deployed in the patient's heart, the radially expanded distal retention portion (12) with the plurality of distal retention segments (22) atraumatically engages the septal wall in the left atrium. With the device (10) in its deployed configuration, each distal retention segment (22) expands in width from its delivery configuration, the distal retention portion (12) extends radially outwardly away from the longitudinal axis of the core region (18), forming a plane at an angle, for example, perpendicular to the longitudinal axis of the core region (18). Upon deployment in vivo, the distal retention portion (12) is configured to be positioned inside the left atrium with each of the distal retention segments (22) opposing the left atrial side of the atrial septum.

In one embodiment of the present teachings, each of the struts (32a, 32b) forming the plurality of distal retention segments (22) join its adjacent struts (32a, 32b) at alternative ends, forming a continuous rounded zigzag pattern with hairpin turns at both ends of the distal retention segments (22), as illustrated in the 2D flat layout in FIG. 2. As shown in the exemplary embodiments, at an elongated delivery configuration, as the two struts (32a, 32b) forming a distal retention segment (22) extend distally from the distal end of the core region (18), they also extend away from each other. As a result, at the free end of each distal retention segment (22), the distance between the two struts (32a, 32b) forming the same distal retention segments (22) is wider than the distance between those same struts near a proximal end of the distal retention segment. Similarly, the distance between the two struts (32a, 32b) forming the same distal retention segments (22) near a proximal end of the distal retention segment (22) is narrower than the distance between those same struts at a free end of the distal retention segment (22). When the device (10) deploys from its delivery configuration, the distal retention segments (22) widen, causing strain on the struts (32a, 32b) near the free end of the distal retention segments (22), and all of the struts (32a, 32b) move radially away from the longitudinal axis (L) of the central core segment (18). And thus, this exemplary configuration is to help to manage strain concentrations at these portions of the device.

According to some embodiments, upon deployment, the distal retention portion (12) forms a disc-like configuration, with at least a portion, sometimes a substantial portion, of the surface area of each retention segment (22) contacting the atrial septum. In other embodiments, the distal retention portion (12) forms an umbrella-like configuration with at least a portion, sometimes a substantial portion, of the surface area of each retention segment (22) doming away from the atrial septum. For example, one or more free ends of the distal retention segments (22) may contacts/contact the atrial septum only at its/their distal free end(s). In yet another embodiment, the distal retention portion (12) forms a generally straight slope profile with at least a portion, sometimes a substantial portion, of the surface area of each distal retention segment (22) not in contact with the atrial septum, so that one or more free ends of the distal retention segments (22) remain(s) further distally away from the proximal ends of the distal retention segments (22). One skilled in the art should understand that other suitable profiles could also be used. Thus the exemplary embodiments discussed, shown, or mentioned herein should not be viewed as limiting.

According to some embodiments, the free end of each distal retention segment (22) includes a foot (52). The foot (52) is configured to prevent the free ends of the distal retention segments (22) from penetrating, piercing, or eroding into the septal tissues. According to some embodiments, the foot (52) is configured to increase surface area for contacting the tissues and/or reducing the pressure that the distal retention segments (22) apply onto the tissues. In some embodiments, the foot (52) is also configured to incorporate a radiopaque marker. For example, a radiopaque marker can be placed into a hole on each of the feet (52).

Similar to the distal retention portion (12), embodiments of the device (10) also have a proximal retention portion (14), joining the proximal end of the core region (18). As shown in the figures, when the device (10) is deployed in position in the patient's heart, the radially expanded proximal retention portion (14) with the plurality of proximal retention segments (24), formed from adjacent struts (34a, 34b) atraumatically engage the septal wall in the right atrium. With the device (10) in its deployed configuration, each of the proximal retention segments (24) expands in width, the proximal retention portion (14) extends radially outward and distally toward the atrial septum forming an umbrella-like profile, as illustrated in FIGS. 1A-1D. As shown in the figures, the proximal end of the proximal retention portion (14) joins the proximal end of the core region (18) of the device, together forming a continuous and smooth curve greater than 90°. As the proximal retention portion (14) extends distally toward the atrial septum, the distal end of the proximal retention portion (14) contacts the atrial septum. Upon the device's deployment in vivo, the proximal retention portion (14) is configured to be positioned inside the right atrium with each of the proximal retention segments (24) opposing the right atrial side of the atrial septum.

In one embodiment of the present teachings, similar to the distal retention portion, the struts forming the plurality of proximal retention segments (24) join its adjacent struts (34a, 34b) at alternative ends forming a continuous rounded zigzag pattern with hairpin turns at both ends of the proximal retention segments (24) as illustrated in the 2D flat layout in FIG. 2.

In one embodiment, the number of distal retention segments (22) is the same as the number of distal retention segments (22). In one embodiment, the number of the distal retention segments (22) is different from the number of distal retention segments (22). In one embodiment, there is an even number of proximal retention segments (24). In one embodiment, there is an odd number of proximal retention segments (24).

Referring to FIGS. 1C-1D, during deployment, the radially outward motion of both distal and proximal retention portions (12, 14) occurs at the ends that join the core region (18), i.e., the section at or near the joint between the core region (18) and the distal retention portion (12), and the section at or near the joint between the core region (18) and the proximal retention portion (14). Consequently, these two joint sections experience a greater strain than the remainder of the distal and proximal retention portions. To minimize any possible impact that these relatively high strain and stress regions may have upon the retention segments (12, 14) during the device deployment such as, for example, turning, twisting, and/or bending motions, exemplary embodiments include a plurality of single transition struts, i.e., distal transition struts (27) and proximal transition struts (30). In embodiments of the present teachings, the turning/bending/twisting during device deployment occurs mostly at the portion of the distal and proximal transition struts (27, 30), thereby allowing the core region (18) to form a tubular profile upon deployment and the distal and proximal retention portions (12, 14) to assume their pre-set deployment profile. The length of the distal transition struts (27) and proximal transition struts (30) may be the same or different. The width and thickness of the distal transition struts (27) and proximal transition struts (30) are configured to more evenly distribute strain over a greater area to avoid undesired deformations and possible device failure.

Although in the exemplary embodiments both the distal and proximal transition struts (27, 30) are illustrated having a generally straight shape, one skilled in the art should understand that these struts may have other shapes and geometries, such as curved profiles with narrow waists. In some embodiments, the proximal transition struts (30) are longer and narrower than the distal transition struts (27). In some embodiments, the proximal transition struts (30) have the same length and/or width as the distal transition struts (27).

In some embodiments, the distal and proximal transition struts (27, 30) have a width from about 0.010″ to about 0.025″. Additionally, in embodiments, the width of the distal transition struts (27) is greater than the thickness to help control bending directions and minimize torsion. One skilled in the art should understand that the length and width of the distal transition struts and proximal transition struts could vary according to the overall size and the intended uses of the device, and thus the exemplary embodiment described here should not be used to limit the spirit of the present teachings.

According to one embodiment of the present teachings, the device is configured to engage atrial septum of varying thickness. Thus, in one embodiment, when the device is deployed in an unconstrained environment, there may be a gap between the distal ends of the proximal retention portion (14) (which extend distally towards the distal retention portion (12)) and the distal retention portion (12). In another embodiment, there is no gap between the distal ends of the proximal retention portion (14) and the distal retention portion (12). In yet another embodiment, the distal end of the proximal retention portion (14) could even protrude and extend distally of the distal retention portion (12). According to one embodiment of the present teachings, the gap between the distal end of the proximal retention portion (14) and the distal retention portion (12) is configured to be greater than the thickness of the atrial septum. As such, in some embodiments, the proximal and distal retention portions (12, 14) may cooperate to apply a compressive force to the septal wall. In another embodiment, the gap between the distal end of the proximal retention portion (14) and the distal retention portion (12) is configured to be similar to the thickness of the atrial septum and therefore the proximal and distal retention portion (12) apply a negligible to no compressive force to the atrial septum.

As disclosed previously, in one embodiment of the present teachings, each of distal and proximal retention segments (22, 24) comprises two struts (32a, 32b, 34a, 34b). According to some embodiments, at least some of the proximal retention segment struts (34a, 34b) are longer than some of the distal retention segment struts (32a, 32b). In other embodiments, all of the proximal retention segment struts (34a, 34b) are longer than the distal retention segment struts (32a, 32b). In some embodiments, the distal retention segment struts (32a, 32b) have a length of about 2-7 mm. In some embodiments, the proximal retention segment struts (34a, 34b) have a length of about 2-14 mm. One skilled in the art should understand that the specific length of the distal retention segment struts (32a, 32b) and/or proximal retention segment struts (34a, 34b) can be determined by, inter alia, the overall size of the device, which in turn is determined by the needs of a patient.

According to some embodiments, the proximal retention segment struts (34a, 34b) have a similar width as the distal retention segment struts (32a, 32b). In other embodiments, the proximal retention segment struts (34a, 34b) have a different width than the distal retention segment struts (32a, 32b). In yet another embodiment, the width of the core region (18) struts (28) is greater than that of the proximal retention segment struts (34a, 34b) and that of the distal retention segment struts (32a, 32b), so that the core region (18) is stiffer than the proximal and distal retention portions (12, 14). According to one embodiment of the present teachings, upon deployment, the stiff core region (18) applies chronic radial outward force to the surrounding tissue, thereby maintaining the size of the opening for the duration of the treatment, while the relative pliable proximal and distal retention portions (12, 14) gently contact the septal tissue without tissue penetration.

In some embodiments, the radial span (i.e., the expanded diameter) of the distal retention portion (12) in the deployed configuration may be the same as the radial span of the proximal retention portion (14). In other embodiments, the radial span of the distal retention portion (12) may be greater than the radial span of the proximal retention portion (14). In some embodiments, the deployed distal retention portion (12) has a radial span of 8-23 mm (or about 15-23 mm, about 17-21 mm, about 18-20 mm, etc.). In another embodiment, the deployed proximal retention portion (14) has a radial span of 8-23 mm (or about 15-23 mm, about 17-23 mm, about 19-23 mm, about 20-22 mm, etc.). According to some embodiments, upon the device's deployment, the diameter of the deployed core region (18) of the device is about 25-50% of the overall radial span of the deployed distal retention portion (12).

With continued reference to FIGS. 1A-1D, the device further comprises a retrieval portion (16). The distal end of the retrieval portion (16) joins the distal end of the proximal retention portion (14). When deployed, the proximal end of the retrieval portion (16) is positioned radially outward of the proximal end of the proximal retention portion (14), while longitudinally aligns with the proximal end of the proximal retention portion (14). According to one embodiment of the present teachings, the retrieval portion (16) comprises a plurality of the retrieval legs (26) that extend radially outward and proximally from the distal end of the proximal retention portion (14). As shown in the figures, the distal end of each retrieval leg (26) joins the distal end of each proximal retention segments (24), forming a continuous and smooth curve of greater than 90°. In embodiments, the distal-most portion of one or more retrieval legs (26) contact the septal tissue. In one embodiment, the tissue contacting portion of the retrieval legs (26) has an enlarged surface area to help prevent any trauma to the tissue. In one embodiment, the proximal end portions of the retrieval legs extend slightly radially inward, such as shown in FIG. 1A-1D.

With continued reference to FIGS. 1A-1D, when the device (10) is in its deployed configuration, the retrieval legs (26) extend proximally and slightly radially inwardly, with the distal ends of the retrieval legs (26) located at a relatively radially outward location, and the proximal ends of the retrieval legs (26) located at a relatively radially inward location. According to some embodiments, in a deployed profile, the distal end of each retrieval leg (26) joins a proximal end of each proximal retention segment (24). In one embodiment, the proximal end portion of the retrieval leg (26) is configured to be radially inward from the distal end of the retrieval leg (26) and radially outward from the distal ends of the proximal retention segments (24). As shown in FIG. 1C, an angle α between the longitudinal axis (L) and the proximal end portion of the retrieval legs (26) can be about 10-80° (or about 20-80°, 30-80°, 20-70, 30-24°, 40-50°, etc.). One skilled in the art should know, although exemplary embodiments described herein and illustrated in figures disclose that the deployed retrieval legs (26) in a slightly curved profile extending from the distal ends to its proximal ends, specific designs of the deployed retrieval leg (26) can be in any profiles that are suitable for the corresponding applications. Thus, the embodiments herein should not be viewed as limiting to the scope of the present teachings.

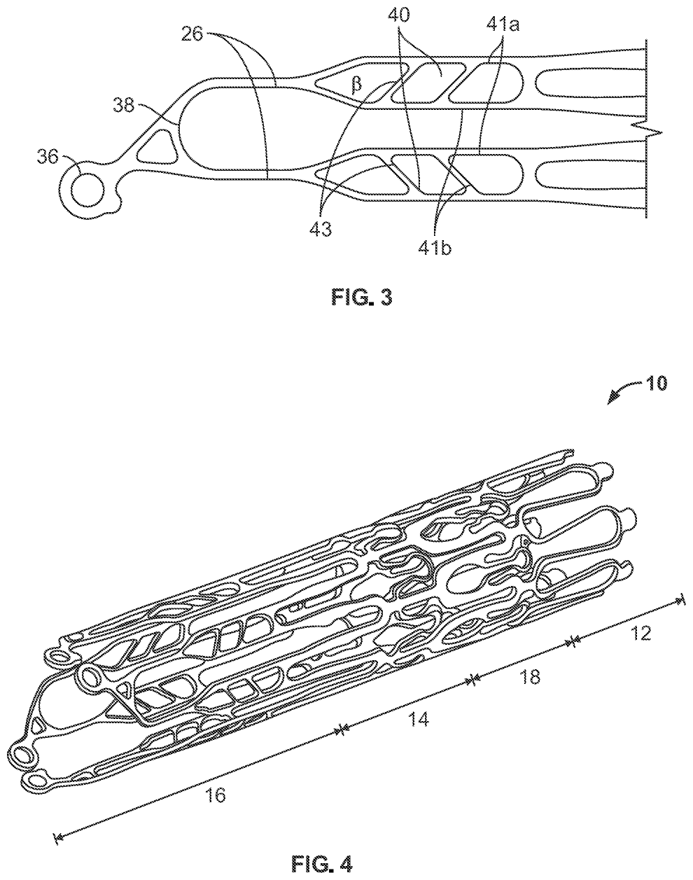

In some embodiments, as shown in FIGS. 1A-1D, two adjacent retrieval legs (26) join near or at their proximal ends. For example. FIGS. 1A and 1B show the two adjacent retrieval legs (26) joining the retrieval eyelet (36) at their proximal ends. In some embodiments, also as illustrated in a close up view of FIG. 3, a connector (38) is placed between adjacent retrieval legs (26) before (distal to) joining at the same retrieval eyelet (36). Connector (38) may prevent connected retrieval legs from interfering with each other during the device delivery, deployment or retrieval.

One skilled in the art should understand that two retrieval legs (26) can join to form a single retrieval leg at any location and a single retrieval leg can continue to extend proximally from that joint location. In yet another embodiment, the retrieval legs (26) do not join with one another. According to some embodiments, as shown in FIGS. 1A-1D, the device (10) includes eight proximal retention segments (24), eight retrieval legs (26), and four retrieval eyelets (36). Each retrieval eyelet (36) joins two adjacent primary retrieval legs (26). In another embodiment, the number of the proximal retention segments (24) and retrieval legs can be any number that is divisible by an integer. For example, there may be nine proximal retention segments (24) and nine retrieval legs (26), and three retrieval eyelets (36), with three adjacent retrieval legs (26) joining the same retrieval eyelets (36). Thus the number of the proximal retention segments (24), retrieval legs (26) and retrieval eyelets (36) illustrated and explained in the exemplary embodiments should not be viewed as limiting.

In some embodiments, the retrieval eyelets (36) are configured to be attached to a flexible delivery mechanism. In some embodiments, a delivery filament, such as a wire or a suture, extends through one or more retrieval eyelet with both ends of the filament being controlled by a clinician. Upon the device's deployment, one end of the delivery filament is loosened and the other end of the delivery filament is retracted proximally so that the entire delivery filament is removed from the body. One skilled in the art would understand that a flexible delivery filament allows the device (10) to fully or partially deploy at a treatment location, while still under the control of the clinician, so that the deployment can be assessed and the device can be retrieved if necessary.

According to one embodiment of the present teachings, the device (10) has an elongated radially collapsed delivery configuration as illustrated in FIG. 4. It should be understood that the flattened view of FIG. 2 is an illustration of the device shown in FIG. 4, but cut and unrolled to a flat configuration. In its delivery configuration, the device has a generally tubular profile, with the distal retention portion (12) radially collapsed with each distal retention segment (22) orienting longitudinally along the longitudinal axis of the core region (18), and the adjacent struts (32a, 32b) (forming each distal retention segment (22)) packed closely and parallelly to one another; the proximal retention portion (14) and the retrieval portion (16) both radially collapsed with the proximal retention segments (24) and retrieval legs (36) also orienting longitudinally along the longitudinal axis of the core region (18), and the adjacent struts (34a, 34b) (forming each proximal retention segment (24)) packed closely and parallelly to one another.

In some embodiments of the present teachings, the device (10) in its delivery configuration has an overall length of about 5-25 mm, with the length of the core region (18) being 0.5-5 mm. In one embodiment, for a deployed device (10), the length of the core region (18) ranges from about 1 mm to about 7 mm, with the overall length of the device (10) ranging from about 3 mm to about 12 mm. In another embodiment, the length of the core region (18) of a deployed device (10) ranges from about 30% to about 70% of the length of the device in the deployed profile.

In some embodiments of the present teachings, the device (10) in its delivery configuration is configured to be delivered and deployed through a 5 French-12 French catheter. In one embodiment, the elongated device (10) has a diameter ranging from about 1 mm to about 4 mm, and the core region (18) in a deployed configuration has a diameter ranging from about 3 mm to about 12 mm, or from about 100% to about 300% of its delivery configuration.

In some embodiments, the device (10) is fabricated from a tube. Thus, all portions of the device (10), such as the distal retention portion (12), the core region (18), the proximal retention portion (14), and retrieval portion (16), have a same thickness. In some embodiments, the thickness of the tube, and thus the thickness of each portion of the device (10), is from 0.003-0.009 inch. In another embodiment, at least one portion of the device (10) has a different thickness than the rest of the device (10). This, in some circumstances, can be achieved by removing material from certain portions.

In some embodiments, the width of the struts throughout the entire device (10) is the same. In another embodiment, the width of the struts forming each portion, such as the distal retention portion (12), core region (18), proximal retention region (14), varies. In some embodiments, the width of the struts throughout the entire device (10) is the same as the thickness of the strut. In another embodiment, the width of the struts is greater than the thickness of the struts in at least one portion of the device (10), such as the proximal retention portion (14), and the retrieval legs (26).

According to one embodiment of the present teachings, the device (10) is pre-set into its deployed profile and stretched into an elongated profile for percutaneous delivery. Upon deployment, the device (10) will recover to its pre-set deployed configuration after it is free from any constraint of the delivery system.

According to one embodiment of the present teachings, the device (10) is delivered through a delivery system for deployment in the atrial septum of the patient's heart. In an embodiment, delivery system includes a delivery catheter (42) and a delivery shaft (44). FIG. 5 illustrates an exemplary embodiment where the device (10) collapses radially into a generally tubular profile and be placed over a delivery shaft (44) and constrained within a delivery catheter (42). As shown in FIG. 5, in this delivery configuration, the distal retention portion, the core region (18), the proximal retention portion (14), and the retrieval portion (16) all collapse radially and orient longitudinally along the longitudinal axis of the core region (18). In some embodiments, the delivery shaft (44) comprises notches (46) for receiving the retrieval eyelets (36). During a delivery, the retrieval eyelet(s) (36) is/are secured inside the notch(es) (46), and, upon deployment, the retrieval eyelet(s) (36) is/are released from the notch(es) (46). In one embodiment, the retrieval eyelets (36) are curved or angled such that they match a slope on the notches (46) of the delivery shaft (44) in order to maintain a smaller device delivery profile. One skilled in the art would understand that a relatively rigid delivery shaft (44) can push the device (10) distally inside the delivery catheter (42) and to deploy device.

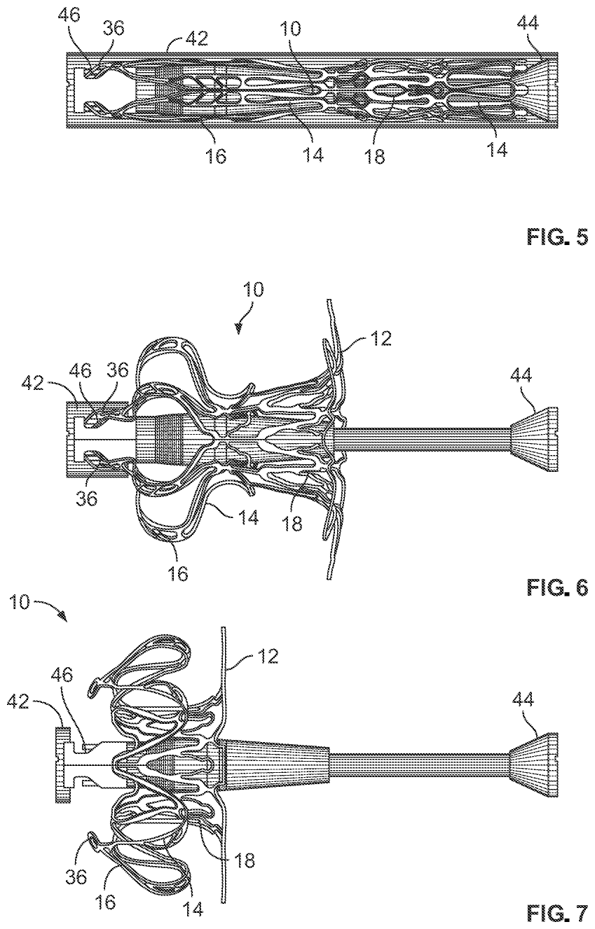

When deploying the device (10) through the septal wall, the delivery system carries a device (10) over a delivery shaft (44), and is advanced through an opening on the septal wall. As the distal end portion of the delivery system is advanced through an opening in the septum and inside the left atrium, while holding the delivery shaft (44) steady, a clinician withdraws delivery catheter (42) proximally to allow the device (10) emerge from the delivery catheter (42). The distal retention portion (12) of the device (10) begins to self-expand inside the left atrium, with the distal retention segments (22) turning radially away from the longitudinal axis of the core region (18) of the device (10). The entire delivery system then is retracted proximally so that the deployed distal retention portions (12) contact the left atrial side of the septal wall. The clinician then holds the delivery shaft (44) steady while further withdraws the delivery catheter (42) proximally to expose the core region (18) and the proximal retention portion (14). The core region (18) and the proximal retention portion (14) expand as they emerge from the delivery catheter (42) with the core region (18) engaging the septal wall opening, and the proximal retention portion (14) contacting the right atrial side septal wall, respectively. At this stage of the deployment, the retrieval eyelets (36) remain attached to the delivery system, i.e., inside the delivery catheter (42) and within the notches (46) on the delivery shaft (44) as shown in FIG. 6. As shown in FIG. 6, the distal retention segments (22) and the core region (18) is substantially expanded into the deployed configurations, the proximal retention segments (24) is partially deployed, and the retrieval legs (26) extend proximally and radially inward with the retrieval eyelets (36) remaining secured by the delivery system. In this stage of the device deployment, since the retrieval eyelets (36) remain attached to the delivery system, as the device (10) deploys, the proximal retention portion (14) expands mostly radially outward with the proximal retention segments (24) turn radially away from the longitudinal axis of the core region (18) of the device (10), and retrieval portion (16) expands with the retrieval legs (26) turning in an opposite radial direction from and radially inward toward the longitudinal axis of the core region (18) of the device (10).

At this stage of the deployment, as shown in FIG. 6, a clinician can assess the device deployment and make a decision of retrieval or release. If device retrieval is needed, the entire device (10) can be pulled back into the delivery system using a combination of distal pushing on the delivery catheter (42) and proximal withdrawal of the delivery shaft (44). The retrieval force applied to the device (10), a result of the combined force of distal push on the delivery catheter (42) and proximal withdrawal of the delivery shaft (44), reverses the radial turn at the joints of retrieval legs (26) and proximal retention segments (24), the radial turn at the joints of proximal retention segments (24) and core region (18), and the radial turn at the joints of core region (18) and distal retention segments (22), forcing the device (10) to enter the distal end of the delivery catheter (42), and resume the elongated profile.

Referring back to FIG. 6, if a clinician is satisfied with device deployment, the device (10) can be released from the delivery system. FIG. 7 illustrates an embodiment of the present teachings, where the device (10) is released from the delivery system. As shown in the figure, the retrieval legs (26) are released from the delivery catheter (42), and move toward their expanded at-rest shapes. Due to being free of any constraint imposed by the delivery system, the proximal retention portion (14) begins to extend distally toward the atrial septum, and the retrieval legs (26) move radially outward to their at-rest positions radially outside of the proximal retention portion (14) of the devices (10).

According to one embodiment, the curved deployment configuration of the proximal retention portion (14) allows the device (10) to accommodate various atrial septum thickness. For example, for a thin atrial septum, the curved proximal retention segments (24) can fully assume its pre-defined curved deployment configuration. For a thick atrial septum, the curved proximal retention segments (24) can oppose the atrial septum, and upon contact, the curved proximal retention segments (24) can deflect at its proximal curved end while maintaining the device (10) in place. In some embodiments, the plurality of proximal retention segments flexes independently of each other to allow the proximal retention portion of the device to accommodate septal walls with uneven thicknesses, for example a septal wall that is thicker near septal secumdum and more narrow near septal primum.

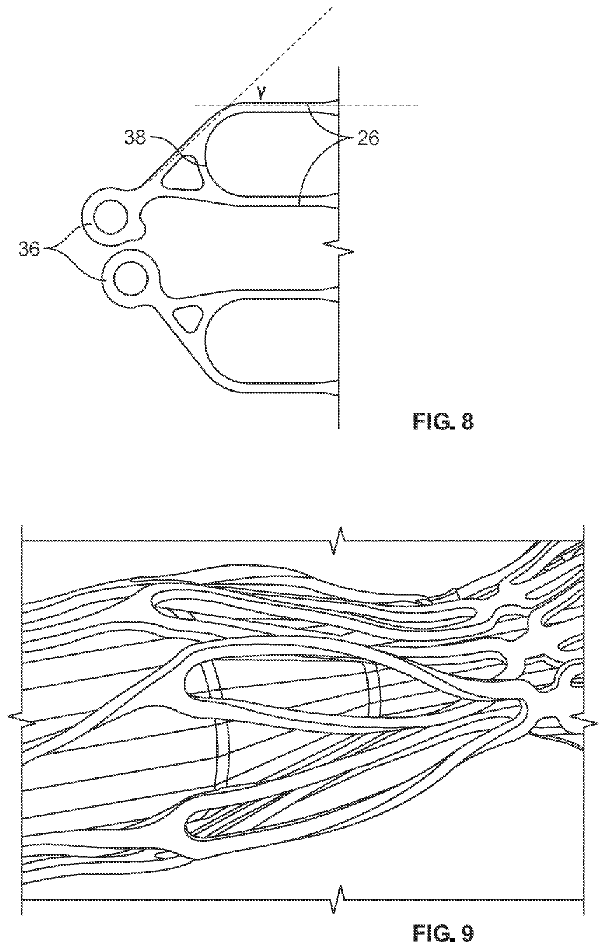

As illustrated previously in FIGS. 1A-1D, the device comprises four retrieval eyelets (36). According to one embodiment of the present teachings, the adjacent eyelets (36) can be paired together, as shown in FIG. 8 when the device is in its delivery profile. As shown in the exemplary flat layout view of FIG. 8, when the device is elongated, one eyelet (36) may extend further proximally than an adjacent eyelet (36). This offset positioning of the two eyelets (36) allows their corresponding retrieval legs (26) to at least partially overlap with each other when being elongated loaded inside the delivery system. The configuration allows a reduction in the overall size of the delivery system whiles maintaining the overall symmetry for the device delivery purpose.

Continuing referring to FIG. 8, each retrieval eyelet (36) joins two adjacent retrieval legs (26). In an embodiment, the retrieval eyelet (36) is configured to be generally aligned with one of the two adjacent retrieval legs (26). In an embodiment, the retrieval eyelet (36) is offset from one or both of the retrieval legs (26) by an angle γ with respect to the longitudinal axis of the device. In some embodiments, the angle γ is between about 30-60°. In another embodiment, the adjacent retrieval eyelet (36) joining two other retrieval legs (26) is configured to be in a generally mirror symmetry configuration, as shown in FIG. 8. While a mirror symmetry configuration is described and illustrated here, it would be readily appreciated that, in some embodiments, two pairs of retrieval legs (26) joining to the two adjacent retrieval eyelet (36) could have different configurations.

According to one embodiment of the present teachings, the retrieval legs (26) of the device (10) could be a single strut extending from the distal end of the proximal retention segment (14) to the retrieval eyelet (36). To help prevent possible twisting of such single struts retrieval legs (26) from torsion which sometimes occurs when the device (10) is loaded inside the delivery system, and, during device deployment and/or retrieval, for example as illustrated in FIG. 9, the width of the single strut retrieval legs (26) may be greater than the radial clearance between the delivery catheter (42) and delivery shaft (44). The radial clearance between the delivery catheter (42) and the delivery shaft (44) is generally the difference between the inner radius of the delivery catheter (42) and the outer radius of the delivery shaft (44). A narrow radial clearance between the delivery catheter (42) and delivery shaft (44) helps to restrain the retrieval legs in their elongated non-twisted configuration.

According to another embodiment of the present teachings, the struts, for retrieval legs (26) and the proximal retention segments (24), are configured to have a low bending stiffness and/or a high torsional stiffness. Torsional stiffness, also known as torsional rigidity, is the ability of an object to resist twisting when acted upon by an external force, torque. The higher the torsional stiffness, the harder for the object to twist under a given load. The lower the torsional stiffness, the easier for the object to twist under the same given load. The torsional stiffness is calculated using the following formula

$$Torsional Stiffness = \frac{GJ}{L}$$

where G is the shear modules, a material property that measures the stiffness of a solid material; J is the polar moment of inertia; and L is the length of the beam. For a beam with a rectangular cross section, J (polar moment of inertia) is calculated as

$$J = \frac{bd ( {b}^{2} + {d}^{2} )}{12}$$

where b is the width of the beam and d is the thickness/depth of the beam. Thus the bending stiffness can be calculated using the following formula

$$Torsional Stiffness = \frac{Gbd ( {b}^{2} + {d}^{2} )}{12 L}$$

Bending stiffness is the amount where a beam will deflect under a given load. The higher the stiffness, the less the deflection that will occur. The lower the bending stiffness, the more the deflection that will occur. In another word, the lower is the bending stiffness, the easier the beam is to bend. Bending stiffness is calculated as

$$Bending Stiffness = \frac{E \cdot I}{L} ,$$

where E is the Youngs Modulus of Elasticity, a material property that measures the ability to withstand changes in length when under tension or compression; I is the Moment of Inertia; and L is the length of the beam. For a beam with a rectangular cross section, I (Moment of Inertia) is calculated as

$$I = \frac{{bd}^{3}}{12}$$

Where b is the width of the beam and d is the thickness/depth of the beam. Thus the bending stiffness can be calculated using the following formula

$$Bending Stiffness = \frac{{Ebd}^{3}}{12 L}$$

According to one embodiment of the present teachings, a device with a high torsional stiffness and a low bending stiffness can be achieved by increasing the strut width, and/or by optimizing the strut width to thickness ratio. Thus, in order to prevent the device distortion during the deployment and retrieval as shown in FIG. 9, in one embodiment of the present teachings, the struts forming the retrieval legs (26) are configured in such way to allow the retrieval legs (26) to have a low bending stiffness so that they readily curve as designed to assume a deployment configuration or resume a delivery configuration without twisting.

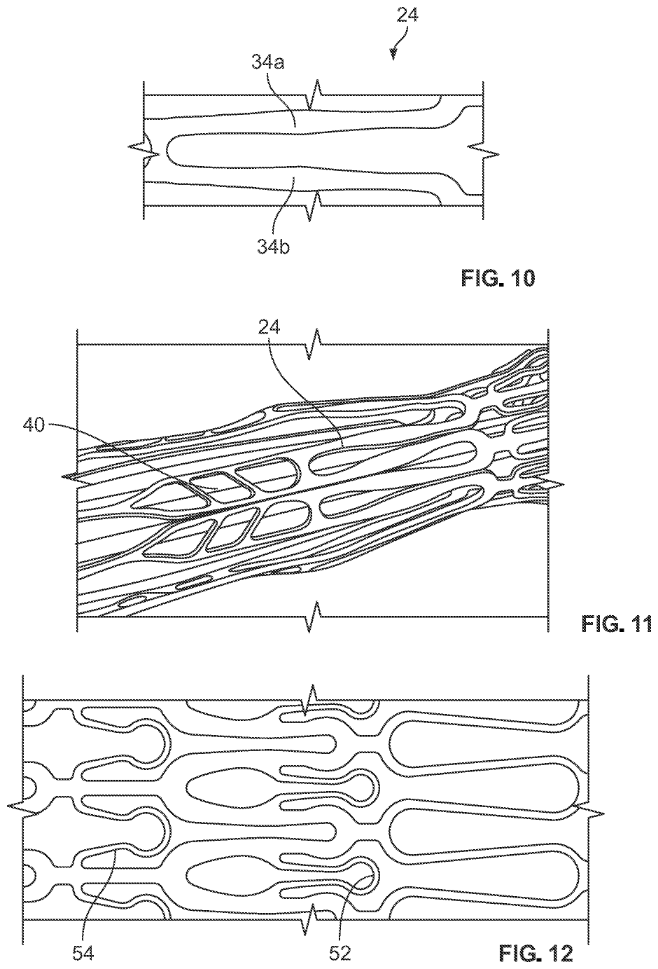

According to one embodiment, optimum bending stiffness and torsional stiffness can be accomplished by uniformly adjusting the strut width and thickness, i.e., maintaining a constant width and thickness throughout the entire length of a strut. In another embodiment, the optimum bending stiffness and torsional stiffness can be accomplished by adjusting the width and thickness/depth of selective portion of the strut, for example as illustrated in FIG. 10. FIG. 10 illustrates a proximal retention segment (24) formed with two adjacent struts (34a, 34b). In this exemplary embodiment, the width of both struts (34a, 34b) gradually increases from one end toward the middle, reaches a maximum width at the middle, followed by a gradual decrease toward the other end. In one embodiment, the width of a middle portion of the struts (34a, 34b) is about 110%-100% of the width of the end portion of the same struts.

In an embodiment, the profiles of the two adjacent struts (34a, 34b) forming a proximal retention segment (24) are identical, i.e., narrowed at both end, and wider in the middle. This configuration increases torsional stiffness to the struts (34a, 34b) and prevents the device (10) from distortion during a deployment and retrieval. In one embodiment, all proximal retention segments (24) share an identical profile. In another embodiment, each proximal retention segment (24) incorporates a different width/thickness ratio and/or a different strut width profile. As such, the exemplary embodiments described herein should not be viewed as limiting to the spirit of the present teaching.

FIG. 3 illustrates another embodiment of retrieval leg (26) configuration resulting in a low bending stiffness and a high torsional stiffness. As shown in FIG. 3, a distal portion of the retrieval legs (26) comprises a ladder portion (40) and a proximal portion of the retrieval leg (26) remains a single strut. The ladder portion (40) of the retrieval leg (26) comprises two generally parallel, longitudinally extending struts (41a, 41b) with one or more connecting segments (43) extending in between. In some embodiments, the one or more connecting segments (43) is positioned at an angle β to the two extending struts (41a, 41b). The angle β can be an angle other than 90° (e.g., 100-150°, 110-140°, 115-135°, etc.). The connecting segments (43) are oriented such that they are non-perpendicular to the extending struts (41a, 41b) in order to prevent the connecting segments (43) from catching a distal end of the delivery catheter (42) when the device (10) is being withdrawn into the delivery system.

Continuing referring to FIG. 3, the distal ladder portions of the two adjacent retrieval legs (26) sharing the same retrieval eyelet (36) have a mirror symmetry design. Accordingly, the exemplary embodiment shown previously in FIG. 1A-1D has four retrieval eyelets (36), and therefore four retrieval leg (26) pairs all with a mirror symmetry distal ladder portion (40) and a single strut proximal portion. In one embodiment, such symmetric pairs design balances the torsional stress imposed upon the device (10) during deployment/retrieval, and thereby maintains a steady and controlled state during device deployment/retrieval, i.e., being loaded back into the delivery catheter (42).

According to one embodiment, the overall width of the ladder portion (40) is greater than the radial clearance between the inner delivery shaft (44) and an inner surface of the delivery catheter (42). In another embodiment, the ladder portion (40) is configured to have a low bending stiffness and a high torsional stiffness so to prevent the retrieval legs from twisting during a deployment and retrieval, as illustrated in FIG. 11.

Now referring to FIG. 12 and FIG. 2, in one embodiment of the present teachings, the device (10) further comprises a plurality of mini struts (52, 54) at or near its core region (18). The use of term “mini struts” (52, 54) refer to the smaller size (e.g., width and/or length) of these struts comparing to those struts that form the distal and proximal retention segments (24) and the core region (18). In this exemplary embodiment, a distal plurality of mini struts (52) is positioned at a distal portion of the core region (18). A proximal plurality of mini struts (54) is positioned at a proximal portion of the core region (18). According to one embodiment of the present teachings, the incorporation of mini struts (52, 54) increases the tissue coverage area of the core region (18), decreases the strut-to-strut opening in the core segment, keeps the surrounding septal tissue from draping into the lumen, and reduces the tissue ingrowth.

In one embodiment, referring to FIG. 2, the addition of the proximal mini struts (54) allows the proximal radially bending portion (A) of the deployed device (10), i.e., a portion of the device (10) from the distal end of the proximal retention segments (14) to the proximal end of the core region (18), to have a consistent total cross sectional area. Similarly, the addition of the distal mini struts (52) allows the distal radially bending portions (B) of the deployed device (10), i.e., a portion of the device (10) from the distal end of the core region (18) to the proximal end of the distal retention segment (12), to have a consistent total cross sectional area. Total cross sectional area is defined as the sum of all cross sectional area of the struts at any given longitudinal section within these two portions. In one embodiment of the present teachings, similar to what has been described earlier, as the device (10) deploys, the distal and proximal retention regions (12, 14) assume their pre-set configuration, both distal and proximal bending portion go through a transformation from relatively straight delivery profile into a relatively curved deployed profile with different radii. In one embodiment, this design configuration reduces the strain concertation and prevents device fatigue.

In one embodiment, the mini struts are configured to achieve a uniform radial stiffness for the core region (18) along its entire length. In one embodiment, the radially stiffness of the core region (18) is constant with no more than 10% variation from its distal end to its proximal end.

In another embodiment, the incorporation of mini struts (52, 54) also improves chronic outward force (COF) and radial resistance force (RRF) that the core region (18) exerts on the surrounding septal tissue. A core region (18) with a strong chronic outward force would be less likely to have device fatigue, and also reduce the need of subsequent ballooning step to for the purpose of restoring blood flow through the core region (18). In one exemplary embodiment, the device (10) is configured to be able to impose a 4.5-5 lbs of chronic outward force (COF) to the surrounding septal tissue when the deployed within a septal opening of 5 mm. In one exemplary embodiment, the device (10) is configured to be able to impose a 1-1.5 lbs of chronic outward force (COF) to the surrounding septal tissue when the deployed within a septal opening of 7 mm. In another embodiment, the device (10) is configured to be able to impose no chronic outward force (COF) to the surrounding septal tissue when the deployed within a septal opening of 8 mm.

In one embodiment, in addition to smaller width, the mini struts (52, 54) also have a reduced thickness compared to the struts that form the distal and proximal retention segments (22, 24) and the core region (18). For example, the mini struts (52, 54) have a thickness of about 0.15-0.25 mm (e.g., about 0.20 mm). In another embodiments, the mini struts (52, 54) have substantially similar thickness to the other portions of the device.

FIG. 12, a flat layout and close-up view of the core region (18) of the device (10), illustrates exemplary placement of the mini struts (52, 54) in accord with some embodiments of the present teachings. As shown, all the mini struts (52, 54) are in a generally hairpin shape with their curved ends distal to the two separate ends. Each of the two separate ends of the mini strut (52, 54) joins each of the two adjacent struts. In one embodiment, each of the two separate ends of the distal mini struts (52) joins each of the two adjacent struts forming the core region (18); and each of the two separate ends of the proximal mini struts (54) joins each of the two adjacent struts forming the distal transition struts (27). In one embodiment, each of the two separate ends of the proximal mini struts (54) joins each of the two adjacent struts forming the proximal retention segments (24). In one embodiment, the curved end of the mini struts (52, 54) could be configured with a larger radii, i.e., greater than a distance between the two elongated portion of the mini-struts in order to provide strain relief and prevent stress concentration. In one embodiment, the joint between the separate ends of the mini struts (52, 54) and the corresponding struts is configured to prevent the mini struts (52, 54) from catching the distal edge of the delivery catheter (42) during a device retrieval. For example, such joint is configured to be non-perpendicular to the longitudinal axis of the core region (18).

In one embodiment, the distal mini-strut (52) joints a mid-portion of the core region (18). In one embodiment, the curved ends of the distal mini-strut (52) are positioned at or near the distal end of the distal transitional struts (27). In one embodiment, the proximal mini-strut (54) joints a mid-portion of the proximal transition strut (30). In one embodiment, the curved ends of the proximal mini-strut (54) is at or near the distal end of the proximal transitional struts (30). In one embodiment, additional mini-struts can be incorporated. In one embodiment, both the distal and proximal mini struts (52, 54) can have various curvature profiles so that the core region (18) has generally even tissue coverage distribution (and even non-tissue coverage distribution) and even chronic outward force (COF) distribution. One skilled in the art should understand the specific number and placement of the mini strut (52, 54) could vary in order to achieve the purpose of the present teachings, i.e., increasing the core region (18) tissue coverage and improving the core region (18) chronic outward force (COF). Thus, the exemplary embodiment shown and described herein should not be viewed as limiting.

Now referring to FIGS. 1C and 1D, during a deployment, the distal mini struts (52) expand radially as the core region (18) expands radially with the curved end portion of the distal mini struts (52) extending slightly radially away from the longitudinal axis of the core region (18). Upon deployment, the distal mini struts (52) are positioned at the distal opening of the core region (18). Similarly, during a deployment, the proximal mini struts (54) also expand radially as the core region (18) expands radially with the separate ends portion of the proximal mini struts (54) extending slightly radially away from the longitudinal axis of the core region (18). Upon deployment, the proximal mini struts (54) are positioned at the proximal opening of the core region (18).

It will also be appreciated by those of skill in the art that references to a structure or feature that is disposed “adjacent” another feature may have portions that overlap or underlie the adjacent feature.

Throughout this specifications and the claims which follow, unless the context requires otherwise, the width of a strut refers to a side-by-side span of the strut along the luminal surface of the device; and the thickness of the a refers to the distance between the exterior luminal surface and interior luminal surface of the device.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A device for implanting into an atrial septum of a patient, the device comprising:
a core region of a tubular profile with a distal end, a proximal end, and a central opening configured to permit blood flow through, wherein the core region is configured to have a uniform radial stiffness along its length;
a distal retention portion joining the distal end of the core region forming a distal bending portion, wherein the distal bending portion is configured to move from a generally straight delivery profile into a curved deployed profile with a first radius, and wherein the distal bending portion has a constant cross sectional area along its length;
a proximal retention portion joining the proximal end of the core region forming a proximal bending portion, wherein the proximal bending portion is configured to move from a generally straight delivery profile into a curved deployed profile with a second radius, and wherein the proximal bending portion has a constant cross sectional area along its length;
wherein the device has a delivery configuration in which each of the distal retention portion, the core region, and the proximal retention portion collapse radially and orient longitudinally along a longitudinal axis of the core region; and
wherein the device has a deployed configuration in which both of the distal and the proximal retention portions extend radially away from the longitudinal axis of the core region and both of the distal and the proximal bending portions are in their curved deployed profile.

2. The device of claim 1, wherein the distal retention portion comprises a plurality of distal retention segments.

3. The device of claim 1, wherein the proximal retention portion comprises a plurality of proximal retention segments.

4. The device of claim 1, further comprising a retrieval portion joining an end of the proximal retention portion.

5. The device of claim 4, wherein the retrieval portion collapses radially and orients longitudinally along a longitudinal axis of the core region when the device is in its delivery configuration.

6. The device of claim 4, wherein the retrieval portion extends radially away from the proximal retention portion with a proximal end of the retrieval portion radially outside of the proximal end of the core region.

7. A device for implanting into an atrial septum of a patient, the device comprising:
a core region with a distal end, a proximal end, and a plurality struts joining their adjacent struts forming a continuous rounded zigzag pattern between the distal and the proximal ends;
a distal retention portion joining the distal end of core region, wherein the distal retention portion comprises a plurality of distal retention segments each formed by two adjacent struts;
a proximal retention portion with a first end joining the proximal end of core region, wherein the proximal retention portion comprises a plurality of proximal retention segments each formed by two adjacent struts;
a retrieval portion having a plurality of retrieval legs joining a second end of the proximal retention portion; and
wherein the plurality of retrieval legs are configured to have a low bending stiffness and a high torsional stiffness to impede the retrieval legs from twisting about a longitudinal axis thereof.

8. The device of claim 7, wherein the two struts forming each proximal retention segment are configured to have a low bending stiffness and a high torsional stiffness.

9. The device of claim 7, wherein at least one of the plurality of retrieval legs has a wider middle section.

10. The device of claim 7, wherein at least one of the two struts forming each proximal retention segment has a wider middle section.

11. The device of claim 7, wherein the at least one of the plurality of retrieval legs has a ladder profiled distal portion.

12. The device of claim 11, wherein at least two adjacent retrieval legs have ladder profiled distal portions in mirror symmetrical configuration to each other.

13. The device of claim 7 further comprises a delivery configuration where both the distal retention portion, the core region, the proximal retention portion, and the retrieval portion collapse radially and orient longitudinally along a longitudinal axis of the core region.

14. The device of claim 7 further comprises a deployed configuration where both the distal and the proximal retention portions extend radially away from the longitudinal axis of the core region, and the retrieval portion extends radially away from the proximal retention portion with a proximal end of the retrieval portion radially outside of the proximal end of the core region.

15. A medical system for implanting into an atrial septum of a patient, the system comprising:
a delivery system having a delivery catheter and a delivery shaft, wherein the delivery catheter comprises a proximal end, a distal end, and an elongated lumen extending from the proximal end of the delivery catheter to the distal end of the delivery catheter;
a device comprising a core region with a distal end, a proximal end, and a plurality struts joining their adjacent struts forming a continuous rounded zigzag pattern between the distal and the proximal ends of the core region; a distal retention portion joining the distal end of core region, wherein the distal retention portion comprises a plurality of distal retention segments each formed by two adjacent struts; a proximal retention portion with a first end joining the proximal end of core region, wherein the proximal retention portion comprises a plurality of proximal retention segments each formed by two adjacent struts; and a retrieval portion having a plurality of retrieval legs joining a second end of the proximal retention portion;
wherein the medical system has a delivery profile in which the device collapses radially and is slidably disposed over the delivery shaft, and the both the radially collapsed device and the delivery shaft slidably disposed within the elongated lumen of the delivery catheter; and
wherein a width of a portion of at least one of the plurality of retrieval legs is greater than a radial clearance between the delivery catheter and the delivery shaft.

16. The device of claim 15, wherein the width of a portion of the at least one struts forming the proximal retention portion is greater than the radial clearance between the delivery catheter and the delivery shaft.

17. The device of claim 15, wherein a core region is configured to have a uniform radial stiffness along its length.

18. The device of claim 15, wherein the plurality of retrieval legs are configured to have a low bending stiffness and a high torsional stiffness to impede the retrieval legs from twisting about a longitudinal axis of the retrieval legs.

19. The device of claim 15 further comprises a delivery configuration where both the distal retention portion, the core region, the proximal retention portion, and the retrieval portion collapse radially and orient longitudinally along a longitudinal axis of the core region.

20. The device of claim 15 further comprises a deployed configuration where both the distal and the proximal retention portions extend radially away from the longitudinal axis of the core region, and the retrieval portion extends radially away from the proximal retention portion with a proximal end of the retrieval portion radially outside of the proximal end of the core region.
